(19) United States
(12) Patent Application Publication
(10) Pub. No.: US 2012/0305804 A1
(43) Pub. Date: Dec. 6, 2012

(54) DOORKNOB STERILIZATION APPARATUS

(76) Inventor: Edward Goldman, Framingham, MA (US)

(21) Appl. No.: 13/153,489

(22) Filed: Jun. 6, 2011

(51) Int. Cl. A61L 2/10

(52) U.S. Cl. 250/492.1

(57) ABSTRACT

A doorknob sterilization apparatus having a housing, an ultraviolet radiation source disposed within the housing, wherein the apparatus is designed to direct a quantity of ultraviolet radiation to an existing doorknob.

### FIELD OF THE INVENTION

The present disclosure relates generally to sterilization, and in particular relates to the sterilization of doorknobs.

### DESCRIPTION OF RELATED ART

As people become more health conscious, it has become increasingly clear that a major source of disease transmission is caused by direct physical contact between people, or indirect physical contact between a person and a surface that an infected person previously came in contact with.

The type and severity of the transmitted diseases may vary, however a wide variety of bacterial and viral infections can be transmitted by indirect or direct contact, including influenza, the common cold, and H1N1, among many others. Moreover, there is a growing concern of the possibility of widespread pandemics of communicable diseases such as dangerous influenza mutations, or anti-bacterial resistant bacterial strains.

One surface that is a major source of disease transmission is a door handle or doorknob. For example, nearly 100% of people who enter a building use the door handle or doorknob on the entrance door. Further nearly 100% of people who use a restroom use the door handle or doorknob on the bathroom door. Thus, the surface of a doorknob is a highly contacted surface and if contaminated by one user, it may spread a disease to a large number of other people.

Existing solutions for sterilizing doorknobs utilize harsh chemicals. These chemicals can have negative health consequences that may be of equal concern to the transmission of disease.

Therefore, what is needed is an apparatus that can effectively and efficiently sterilize a doorknob, without the use of harsh, dangerous chemicals.

### SUMMARY OF THE INVENTION

The subject matter of this application may involve, in some cases, interrelated products, alternative solutions to a particular problem, and/or a plurality of different uses of a single system or article.

In one aspect, a doorknob sterilization apparatus is provided comprising a housing, defining an aperture at a bottom of the housing, the aperture forming an outlet to the housing, an ultraviolet radiation source disposed within the aperture of the housing, a mounting bracket attached to a rear of the housing, wherein the apparatus is constructed and arranged to direct a quantity of ultraviolet radiation to an existing doorknob.

In another aspect, a doorknob sterilization apparatus is provided comprising a first housing defining an aperture at a bottom of the first housing, the aperture forming an outlet to the first housing, a first ultraviolet radiation source disposed within the aperture of the first housing, a first mounting bracket attached to the rear of the first housing, second housing defining an aperture at a bottom of the second housing, the aperture forming an outlet to the second housing, a second ultraviolet radiation source disposed within the aperture of the second housing, a second mounting bracket attached to the rear of the second housing, wherein the bottom of the first housing is substantially facing the bottom of the second housing, wherein the first housing and the second housing are placed adjacent to an existing doorknob on substantially opposite sides of the doorknob, wherein the first housing is in electronic communication with the second housing, and wherein the apparatus is constructed and arranged to direct a quantity of ultraviolet radiation to an existing doorknob.

In yet another aspect, a doorknob sterilization apparatus is provided comprising a housing defining an aperture at a bottom of the housing, the aperture forming an outlet to the housing, an ultraviolet radiation source disposed within the aperture of the housing, a mounting bracket attached to a rear of the housing, a second housing, a reflector positioned on a surface of the second housing, wherein the reflector of the second housing is positioned to substantially face the ultraviolet radiation source, and wherein the housing and the second housing are positioned about an existing doorknob such that the apparatus is constructed and arranged to direct a quantity of ultraviolet radiation to the existing doorknob.

### BRIEF DESCRIPTION OF THE DRAWINGS

FIG. 1 provides an illustration of an embodiment of the doorknob sterilization apparatus.

FIG. 2 provides an illustration an embodiment of the doorknob sterilization apparatus with a closed front face, thereby hiding the internal components.

FIG. 3 provides an illustration of a side view of an embodiment of the doorknob sterilization apparatus.

FIG. 4 provides an illustration of another embodiment of the doorknob sterilization apparatus.

FIG. 5 provides an illustration of another embodiment of the doorknob sterilization apparatus.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Generally, the present invention provides a sterilizing apparatus that utilizes ultraviolet (UV) radiation to sterilize a doorknob or door handle. The apparatus has a housing, which provides structure to the apparatus, contains the components of the apparatus, and directs the UV radiation at a doorknob surface. This apparatus is designed to operate with existing doorknobs, and as such may be mounted to any door or door jam to direct UV radiation at an existing doorknob. The design of this apparatus therefore provides a low cost, flexible disinfection solution that can be easily incorporated into existing building structure.

The apparatus may be used on any doorknob that is desired to be disinfected. In particular, the apparatus may be particularly useful for public restrooms, public building entrances, offices, schools, and the like. Further, because of its easily installable nature, the apparatus is widely accessible to all potential users regardless of technical skill.

The term doorknob used herein is intended to refer to traditional doorknobs, and also to door handles, door latches, metallic push pads, and generally any structure that may be used to open and close a door.

The doorknob sterilization apparatus may have a housing that provides structure for the apparatus. This housing may be designed to mount to a door or a door frame. The housing may be formed to define an aperture that houses the internal components of the apparatus, thus forming an interior and exterior surface of the housing. The aperture may open to the outside of the housing at a bottom portion of the housing, forming an outlet to the housing. This outlet provides, among other things, an outlet for the UV radiation.

The housing may be positioned in any relationship with a doorknob such that the housing may direct a sufficient quantity of UV radiation to sterilize a surface of the doorknob. Preferably this positioning may be 6-8 inches from the surface of the doorknob.

The housing may be of any shape and size that may direct a sufficient quantity of UV radiation to sterilize a surface of the doorknob. Preferably the housing has a width slightly greater than the largest width of the doorknob, and a length sufficient to store internal components of the apparatus. In one embodiment, the width of the apparatus is approximately 2.75 inches and the height of the apparatus is approximately 4 inches.

The housing may be constructed of any material that may be mounted to a door, that is substantially impermeable to UV radiation, and that is capable of directing a quantity of UV radiation to sterilize a surface of the doorknob. Materials of which the housing can be made include, but are not limited to metals such as stainless steel, steel, brass, aluminum, titanium, tin, copper, and the like, plastics, wood, or composite material. The housing may be manufactured using methods known to those skilled in the art such as bending sheet metal, molding metal, molding plastics and the like.

The housing may serve to house the various components of the sterilization apparatus. These components may include an electronic control board, a timer, a UV radiation source, a reflector, a battery compartment, a motion sensor, and a mounting bracket. In one embodiment, the aperture of the housing may be accessed by a first portion of the housing being hingedly attached to a second portion of the housing. When the housing portion is opened via the hinged attachment, the aperture and components disposed therein may be accessible.

The doorknob sterilization apparatus may have a control board located within the aperture defined by the housing. Preferably the control board may be attached to the inner surface of the housing. The control board may operate to control the electronic components of the apparatus. Further, the control board provides electronic communication between all components that it is in electronic communication with. Parameters that the control board may control may include controlling electricity flow from an electricity source to the UV radiation source, turning the UV radiation source on and off based on operation of a timer, and turning the UV radiation source on and off based on an activation or deactivation from a motion sensor or light sensor.

The control board may have various user input devices including but not limited to timer adjustment, time interval adjustment, an on/off switch, a day/24 hour switch a reset button, and the like. The user input devices may be positioned on the housing in a way that they may be easily accessed by a user, and are in electronic communication with the control board.

The control board may further be in electronic communication with a digital display. The control board may be configured to provide visual readouts of various readings relating to the status of the doorknob sterilization apparatus. For example, the digital display may be configured to display information relating to battery life, the current timer and interval setting, and an indicator that the UV radiation source is activated.

In one embodiment, the timer is set to activate the UV radiation source for ten seconds at five minute intervals. In another embodiment, the timer is set to activate the UV radiation source for twelve seconds at five minute intervals. In still another embodiment, the UV radiation source activation time and interval may be adjustable based on user preference, and considerations such as frequency of door use, door location, and the like. In yet another embodiment, the timer may be connected to a multiple way switch which can allow a user to change the UV radiation source activation time between multiple different time settings.

A UV radiation source may be mounted within the aperture defined by the housing. Preferably the UV radiation source may be oriented to deliver an optimum amount of UV radiation to a doorknob surface.

In one embodiment, the UV radiation source may be a fluorescent bulb. In a further embodiment, this bulb may be a three watt fluorescent bulb. In another embodiment, the UV radiation source may be a light emitting diode (LED). In still another embodiment, the UV radiation source may be configured to produce and direct UV radiation unidirectionally. In another embodiment, the UV radiation source may be configured to produce UV-C radiation which is particularly effective in the destruction of microorganisms.

The UV radiation source may be electrically connected to a source of electricity. In one embodiment, the UV radiation source may be in electronic communication with the control board, which in turn is in electronic communication with a source of electricity. In another embodiment a switch is interposed between the UV radiation source and the source of electricity. In still another embodiment the UV radiation source is directly connected to the source of electricity.

In a further embodiment, a polarizing filter or other device may be employed to limit the scatter of the UV radiation source. The polarizing filter may aid in directing the UV radiation directly at the doorknob, thereby limiting UV radiation exposure to individuals who may be nearby.

A reflector may be disposed within the aperture defined by the housing. The reflector may be in close proximity to the UV radiation source. In one embodiment, the reflector may be located above the UV radiation source to reflect and focus any UV radiation that reaches the reflector towards a surface of a doorknob. The reflector may be made out of any material that may reflect UV radiation. For example, the reflector may be made of polished metal, glass, reflective plastic, Mylar®, and the like. In one embodiment the reflector is disposed in close proximity to the UV radiation source. In another embodiment, the reflector may be attached to the inner surface of the housing, such that the entire inner surface of the housing may act as a reflector.

A battery compartment may be disposed within the aperture of the housing. This battery compartment may be sized to fit a battery or batteries sufficient to power the UV radiation source. In one embodiment a single nine volt battery may be used. In another embodiment, four ‘AA’ type batteries may be used.

In an alternative embodiment, the battery compartment may be formed as a depression in an outer surface of the housing. This embodiment may further include a battery compartment cover, which may cover the batteries, but allow them to be easily accessed and replaced. In one embodiment, the cover may be removably attached to the battery compartment. In another embodiment the cover may be hingedly attached to the battery compartment. In another embodiment, the cover may snap-fit onto the battery compartment.

When a battery or plurality of batteries is positioned within the battery compartment, they may act as a source of electricity, and may be in electronic communication with the control board, switch, or UV radiation source.

The doorknob sterilization apparatus may further comprise a motion sensor. The motion sensor may be positioned anywhere on an outside of the housing. In one embodiment, the motion sensor may be positioned such that it may detect the motion of an approaching person. The motion sensor may be in electronic communication with the control board. In one embodiment the motion sensor may be configured to deactivate the UV radiation source when motion is detected. This embodiment acts as a safety feature to prevent human exposure to a quantity of UV radiation from the UV radiation source. In one embodiment, the motion sensor may measure motion of bodies in view of the motion sensor, thereby detecting motion of people, animals and the like in proximity to the apparatus.

In another embodiment, the motion sensor may be an accelerometer, thereby detecting motion of the apparatus itself. The accelerometer may be positioned anywhere on an outer surface of the housing, or within the aperture formed by the housing. The accelerometer may be in electronic communication with the control board. In one embodiment, the accelerometer may be configured to provide a signal to the control board when it detects that the apparatus is in motion, which in turn may deactivate the UV radiation source. This embodiment acts as a safety feature to prevent human exposure to a quantity of UV radiation from the UV radiation source, for example, when the door is being opened or closed, or when the apparatus is being attached or removed from a door or doorframe.

In yet another embodiment, the motion sensor may utilize both motion of the apparatus with an accelerometer, and also motion of bodies outside the apparatus in view of the motion sensor.

The doorknob sterilization apparatus may further comprise a light sensor in electronic communication with the control board. The light sensor may allow the unit to be used at specific day or night settings. If the apparatus is set to only sterilize during the day, the light sensor may send a signal to the control board that activates the apparatus when the light sensor detects light. Alternatively, if the apparatus is set to only sterilize at night, the light sensor may send a signal to the control board that activates the apparatus when the light sensor does not detect light. This setting may be particularly applicable to non-household usage. For example, if a business employs these units on office doors, it may increase efficiency and save electricity to only operate the apparatus when the lights are turned on in the office.

The doorknob sterilization apparatus may include a mounting bracket. The mounting bracket may be positioned anywhere on the outside of the housing such that the housing may be mounted to a door or door frame to direct UV radiation upon a doorknob. In one embodiment this mounting may be permanent. In another embodiment, the housing may be removably mounted.

In one embodiment the mounting bracket may utilize an adhesive to mount the housing. The adhesive may be any substance that may form a secure connection between the housing and the door or door frame.

In another embodiment, the mounting bracket may utilize screws to mount the housing. The screws may be passed through an aperture formed by the housing, and driven into the mounting surface. This embodiment may utilize nails, sheetrock anchors, or other similar fasteners as well.

The doorknob sterilization apparatus may further comprise a second housing. The second housing may be designed to be positioned to provide UV radiation to any portion of a doorknob that may not receive UV radiation from the UV radiation source of the housing.

In one embodiment, the second housing may comprise the same elements, components and structure of the first housing. In this embodiment, the doorknob sterilization apparatus may comprise two pieces, each piece designed to be positioned opposite to the other to provide UV radiation to all faces of a doorknob.

In another embodiment, the second housing may comprise a reflector. The reflector may be constructed to reflect UV radiation emanating from the housing that passes over the doorknob. In one embodiment, the reflector may be specifically designed to reflect UV radiation emanating from the housing directly at faces of the doorknob that are not contacted with UV radiation from the UV radiation source of the housing.

In still another embodiment, the second housing may comprise a UV radiation source, a reflector, a mounting bracket, a connection port and a second motion sensor that may be in electronic communication with the control board of the housing. This embodiment may utilize the electricity source and control board of the housing to control components of both the housing and the second housing. The second housing may be physically connected to the housing, or may be in electronic communication with the control board of the housing wirelessly.

The second housing may have a second UV radiation source to supplement the UV radiation source of the housing. This second UV radiation source may be positioned within an aperture of the second housing. The second UV radiation source may be in electronic communication with the control board, such that it may be activated and deactivated based on a timer of the control board, a motion sensor of the control board, and the like. In one embodiment, the second UV radiation source may be activated simultaneously with the UV radiation source. In another embodiment, the UV radiation source and second UV radiation source may be activated at different times.

The second housing may further comprise a reflector disposed within the aperture formed by the second housing. The reflector may be located behind the UV radiation source to reflect and focus any UV radiation that reaches the reflector towards a surface of a doorknob. The reflector may be made out of any material that may reflect UV radiation. For example, the reflector may be made of polished metal, glass, reflective plastic, Mylar®, and the like. In one embodiment the reflector is disposed in close proximity to the second UV radiation source. In another embodiment, the reflector may be attached to the inner surface of the second housing, such that the entire inner surface of the second housing may act as a reflector.

The second housing may further comprise a connection port. The connection port may be constructed in any fashion that allows electronic communication between the housing and the second housing. The connection port may be constructed to allow electronic communication between the control board and the second UV radiation source. In one embodiment, a cable may pass between the control board and the second housing. In another embodiment wireless communication may be employed. In still another embodiment, the housing and second housing may be slideably disposed upon a channel that includes an electronic communication between the control board and the second UV radiation source. The channel may provide an adjustable distance between the housing and second housing, and as such may allow the doorknob sterilization apparatus to fit around various doorknob sizes and configurations.

The second housing may further comprise a second motion sensor. The second motion sensor may be positioned anywhere on an outside of the second housing. In one embodiment, the second motion sensor may be positioned such that it may detect the motion of an approaching person. The second motion sensor may be in electronic communication with the control board. In one embodiment the second motion sensor may be configured to deactivate the second UV radiation source when motion is detected. In another embodiment, the second motion sensor may be configured to deactivate both the UV radiation source and the second UV radiation source when motion is detected. These embodiments act as a safety feature to prevent human exposure to a potentially dangerous quantity of UV radiation from the UV radiation source.

In one embodiment of operation, the first and second housings may be mounted to a door or door jam on substantially opposite sides of a doorknob. In a further embodiment, the first and second housing are placed substantially adjacent to the existing doorknob to provide sufficient UV radiation to the existing doorknob. In this embodiment, a bottom of the first housing and a bottom of the second housing may be positioned to be substantially facing each other.

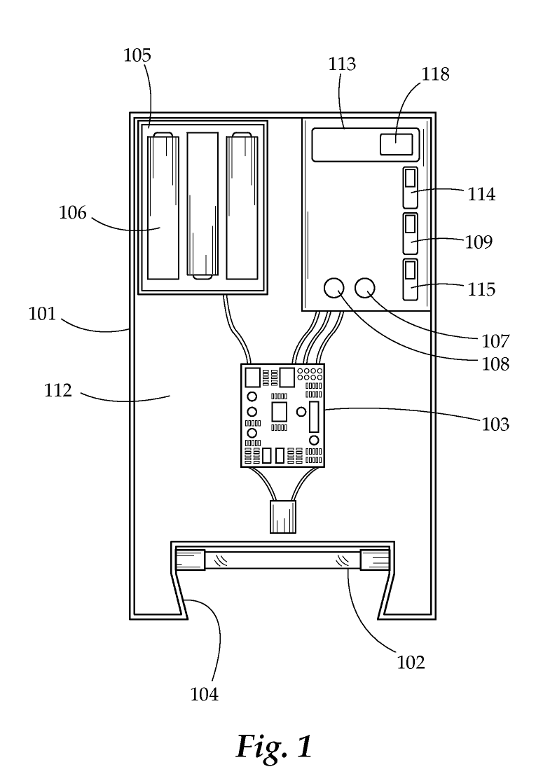

Turning now to FIG. 1, an embodiment of the doorknob sterilization apparatus is shown. The embodiment shown is intended to be positioned above a doorknob, and alternatively may have a second apparatus that mirrors that shown in FIG. 1 mounted below the doorknob. The housing 101 defines the aperture 112 which serves as a storage place for the internal components of the apparatus. The front cover (not shown) of the apparatus is cut away to show the internal components. A bulb 102 is located within the aperture 112 of the housing 101. The bulb 102 is configured to direct ultraviolet radiation downward and out of the apparatus, towards a doorknob (not shown). A reflector 104 surrounds the bulb 102 to direct a maximum amount of ultraviolet radiation out of the apparatus. A battery compartment 105 stores batteries 106, which provide electricity to the apparatus. The batteries 106 are in electronic communication with a control board 103.

The embodiment shown in FIG. 1 has three switches 114, 109, 115 located within the housing 101. All three switches 114,109,115 are in electronic communication with the control board 103. A day/24 hour switch 109, toggles the operation of the apparatus from operating for during the daylight (or office light) hours, or 24 hours a day. The position of the day/24 hour switch 109 may be determined based on the type of usage needs of the doorknob that the apparatus is intended to sterilize. An on/off switch 114 allows the apparatus to be manually turned on and off, depending on the sterilization needs where the apparatus is located. An interval switch 115 may have a number of different time settings to trigger activation of the bulb 102. For example, the interval switch 115 may have settings for 30, 60 and 90 seconds, meaning that there is that many seconds between each bulb 102 activation cycle. The interval may vary depending on the sterilization needs of where the apparatus is located.

The apparatus further has a digital display 113 in electronic communication with the control board 103. The digital display 113 provides a visual display showing the status of the sterilization apparatus. The digital display 113 shows a variety of status indicators, including when the apparatus is on, what settings it is set for, and battery level. The battery level is shown on the battery indicator 118 of the digital display 113.

The apparatus further has a motion sensor 107 and a light sensor 108, positioned within the apparatus, but in visual communication with the outside of the apparatus. The motion sensor 107 is positioned such that it may sense the motion of bodies nearby the apparatus. The motion sensor 107 is in electronic communication with the control board 103. In the event that the motion sensor 107 senses a significant level of motion, the bulb 102 will be shut off if it is activated.

The light sensor 108 is positioned such that it may sense a level of light within its environment. The light sensor is in electronic communication with the control board 103. In the event that the day/24 hour switch 109 is set to day operation, the light sensor 108 will deactivate the apparatus when little to no light is detected.

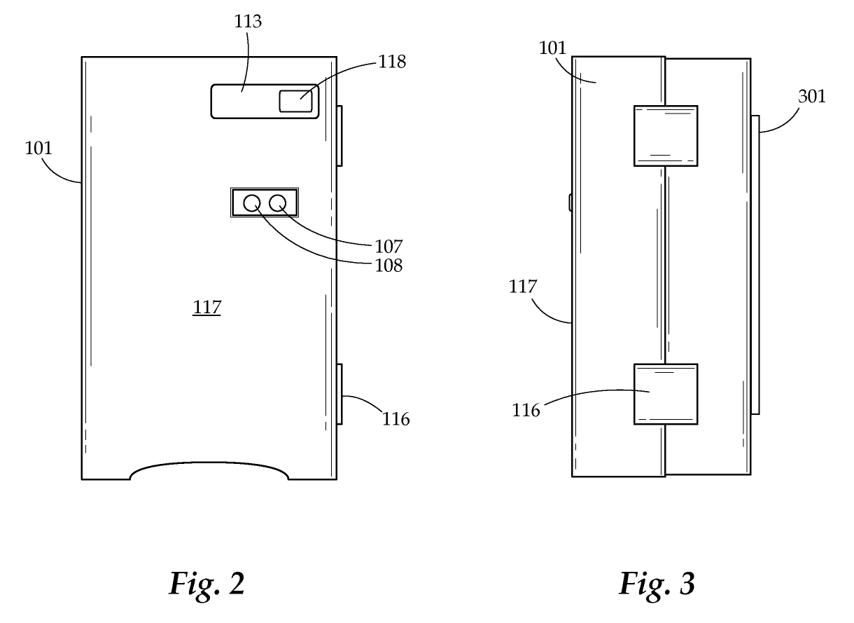

FIG. 2 shows an embodiment of the doorknob sterilization apparatus with a front face 117 closed, thereby hiding the internal components. The apparatus has a housing 101 which provides structure for the apparatus. The housing 101 has a front face 117 hingedly connected to it via hinges 116, allowing the front face 117 to swing from an open to closed position. The front face 117, when closed, covers the internal components and provides an aesthetically pleasing cover for the apparatus. The front face 117 defines a number of apertures that facilitate operation of the apparatus. The digital display 113 is visible through one of these apertures, allowing a user to view the status of the apparatus without opening the front face 117. Shown on this digital display 113 is a battery indicator 118. The motion sensor 107 and light sensor 108 may be positioned to fit through an aperture of the front face 117 such that they can view motion and light on the outside of the apparatus.

FIG. 3 provides a side view of an embodiment of the doorknob sterilization apparatus. This view shows the front face 117 hingedly connected to the housing 101 by hinges 116. Further, the apparatus has a mounting bracket 301 which allows the apparatus to be mounted to doors or doorframes.

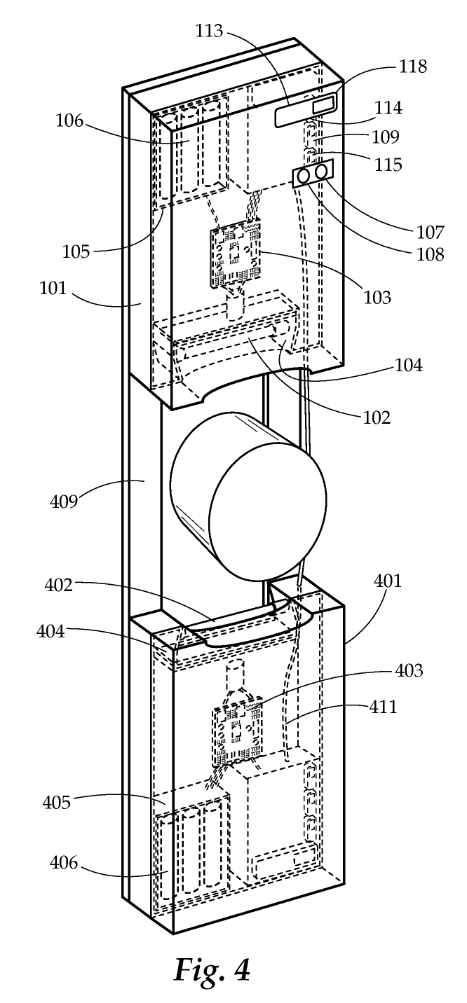

FIG. 4 provides another embodiment of the doorknob sterilization apparatus having two housings in electronic communication with each other. In this embodiment, the first housing 101 and second housing 401 are slideably connected to a track 409 which allows the distance between the housings 101, 401 to be adjusted. This embodiment allows the components of the first housing 101 and the second housing 401 to be in electronic communication via a wire 411. Therefore, the bulb 102 and second bulb 402 may both be controlled by switches on the first housing 101, which communicates operation to components of both the first housing 101 and the second housing 401. Further, this embodiment provides a digital display 113 located in the first housing 101 that displays the status of components located in the first housing 101 and the second housing 401.

A bulb 102 is located within the first housing 101. The bulb 102 is configured to direct ultraviolet radiation downward and out of the apparatus, towards a doorknob (not shown). A reflector 104 surrounds the bulb 102 to direct a maximum amount of ultraviolet radiation out of the apparatus. A battery compartment 105 stores batteries 106, which may provide electricity to the apparatus. The batteries 106 are in electronic communication with the control board 103.

The embodiment shown in FIG. 4 has three switches 114, 109, 115 located within the housing 101. All three switches 114,109,115 are in electronic communication with the control board 103. A day/24 hour switch 109, toggles the operation of the apparatus from operating for during the daylight (or office light) hours, or 24 hours a day. The position of the day/24 hour switch 109 may be determined based on the type of usage needs of the doorknob that the apparatus is intended to sterilize. An on/off switch 114 allows the apparatus to be manually turned on and off, depending on the sterilization needs where the apparatus is located. An interval switch 115 may have a number of different time settings to trigger activation of the bulb 102. For example, the interval switch 115 may have settings for 30, 60 and 90 seconds, meaning that there is that many seconds between each bulb 102 activation cycle. The interval may vary depending on the sterilization needs of where the apparatus is located.

The apparatus further has a digital display 113 in electronic communication with the control board 103. The digital display 113 provides a visual display showing the status of the sterilization apparatus. The digital display 113 shows a variety of status indicators, including when the apparatus is on, what settings it is set for, and battery level. The battery level is shown on the battery indicator 118 of the digital display 113.

The apparatus further has a motion sensor 107 and light sensor 108, positioned within the first housing 101, but in visual communication with the outside of the apparatus. The motion sensor 107 is positioned such that it may sense the motion of bodies near the apparatus. The motion sensor 107 is in electronic communication with the control board 103. In the event that the motion sensor 107 senses a significant level of motion, the bulb 102 and second bulb 402 will be shut off if it is activated. The light sensor 108 is positioned such that it may sense a level of light within its environment. The light sensor is in electronic communication with the control board 103. In the event that the day/24 hour switch 109 is set to day operation, the light sensor 108 will deactivate the apparatus when little to no light is detected.

The second housing 401 is located below a doorknob and is rotated 180 degrees relative to the first housing 101. The front cover (not shown) of the apparatus is cut away to show the internal components. A bulb 402 is located within the second housing 401. The bulb 402 is configured to direct ultraviolet radiation downward and out of the apparatus, towards a doorknob (not shown). A reflector 404 surrounds the bulb 402 to direct a maximum amount of ultraviolet radiation out of the apparatus. A battery compartment 405 stores batteries 406, which may provide electricity to the apparatus. The batteries 106 are in electronic communication with the control board 403. Between the first housing 101 and the second housing 401 there is a power connection 411 that provides electronic communication between the control board 103 of the first housing and the control board 403 of the second housing. This power connection 411 allows the switch settings, motion sensor input, and light sensor input to adjust activation of the components in the first housing 101 and the second housing 401.

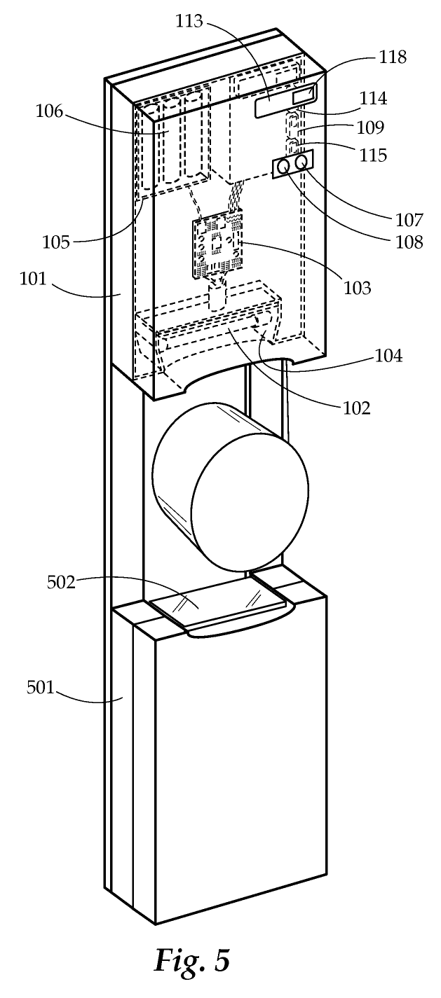

FIG. 5 shows another embodiment of the doorknob sterilization apparatus having two housings. In this embodiment, the first housing 101, and elements therein, is identical to the embodiment of the apparatus shown in FIG. 4. The second housing 501 is positioned substantially opposite to the first housing 101 and is shaped to have an outer appearance similar to the first housing 101. The second housing has a reflector 502 directed to be facing the bulb 102 of the first housing 101. This reflector 502 is positioned to reflect UV radiation emitted from the bulb 102 of the first housing 101 at an underside of a doorknob.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A doorknob sterilization apparatus comprising:
a housing;
an ultraviolet radiation source disposed within an aperture of the housing, and positioned to direct a quantity of ultraviolet radiation out of the housing;
a mounting bracket attached to a rear of the housing; and
wherein the apparatus is constructed and arranged to direct the quantity of ultraviolet radiation to an existing doorknob.

2. The doorknob sterilization apparatus of claim 1 further comprising a control board, in electronic communication with a power source and the ultraviolet radiation source, and providing electronic communication between the power source and the ultraviolet radiation source.

3. The doorknob sterilization apparatus of claim 1 wherein the mounting bracket of the housing is mounted to a door, the existing doorknob being attached to the door.

4. The doorknob sterilization apparatus of claim 2 further comprising a motion sensor attached to an outer surface of the housing and in electronic communication with the control board.

5. The doorknob sterilization apparatus of claim 4 wherein the ultraviolet radiation source is a fluorescent bulb.

6. The doorknob sterilization apparatus of claim 4 wherein the ultraviolet radiation source provides ultraviolet radiation in the UVC range.

7. The doorknob sterilization apparatus of claim 4 further comprising a reflector disposed within the aperture of the housing.

8. The doorknob sterilization apparatus of claim 4 wherein the power source is a battery.

9. The doorknob sterilization apparatus of claim 8 wherein the battery is secured within a battery compartment, the battery compartment disposed within the aperture of the housing.

10. The doorknob sterilization apparatus of claim 9 wherein a front portion of the housing is hingedly attached to a second portion of the housing, and wherein the front portion covers the aperture in a first position and allows access to the aperture in a second position.

11. The doorknob sterilization apparatus of claim 10 further comprising an adjustable timer in electronic communication with the control board, constructed and arranged to activate and deactivate the ultraviolet radiation source at specific time periods.

12. The doorknob sterilization apparatus of claim 11 further comprising a digital display positioned on the housing and in electronic communication with the control unit.

13. The doorknob sterilization apparatus of claim 12 further comprising a user input device in electronic communication with the control board.

14. The doorknob sterilization apparatus of claim 12 further comprising:
a second housing;
a second ultraviolet radiation source disposed within an aperture of the second housing, and positioned to direct a quantity of ultraviolet radiation out of the second housing;
a second mounting bracket, attached to a rear of the second housing;
wherein the second housing is constructed and arranged to direct the quantity of ultraviolet radiation to an existing doorknob; and
wherein the existing doorknob is positioned between the housing and the second housing.

15. The doorknob sterilization apparatus of claim 12 further comprising:
a second housing;
a reflector positioned on a surface of the second housing;
wherein the reflector of the second housing is positioned to face the ultraviolet light source; and
wherein the existing doorknob is positioned between the housing and the second housing.

16. A doorknob sterilization apparatus comprising:
a first housing defining an aperture at a bottom of the first housing, the aperture forming an outlet to the first housing;
a first ultraviolet radiation source disposed within the aperture of the first housing;
a first mounting bracket attached to a rear of the first housing;
a second housing defining an aperture at a bottom of the second housing, the aperture forming an outlet to the second housing;
a second ultraviolet radiation source disposed within the aperture of the second housing;
a second mounting bracket attached to the rear of the second housing;
wherein the bottom of the first housing is substantially facing the bottom of the second housing;
wherein the first housing and the second housing are placed adjacent to an existing doorknob on substantially opposite sides of the doorknob;
wherein the first housing is in electronic communication with the second housing; and
wherein the apparatus is constructed and arranged to direct a quantity of ultraviolet radiation to an existing doorknob.

17. The doorknob sterilization apparatus of claim 16 further comprising:
a control board located within the first housing, the control board providing electronic communication between the first ultraviolet radiation source, the second ultraviolet radiation source, and a power source;
a first motion sensor disposed on an outer surface of the first housing, and in electronic communication with the control board; and
a timer in electronic communication with the control board, constructed and arranged to activate and deactivate the first ultraviolet radiation source and the second ultraviolet radiation source at specific time intervals.

18. The doorknob sterilization apparatus of claim 17 further comprising a track slideably connected to the first housing and the second housing, the track acting as a connection port between the first housing and the second housing, and providing electronic communication between the first housing and the second housing.

19. The doorknob sterilization apparatus of claim 18 further comprising:
a digital display positioned on the first housing and in electronic communication with the control unit; and
a user input device positioned on the first housing, in electronic communication with the control board.

20. A doorknob sterilization apparatus comprising:
a housing defining an aperture at a bottom of the housing, the aperture forming an outlet to the housing;
an ultraviolet radiation source disposed within the aperture of the housing;
a mounting bracket attached to a rear of the housing;
a second housing;
a reflector positioned on a surface of the second housing;
wherein the reflector of the second housing is positioned to substantially face the ultraviolet radiation source; and
wherein the housing and the second housing are positioned about an existing doorknob such that the ultraviolet radiation source is constructed and arranged to direct a quantity of ultraviolet radiation to the existing doorknob; and
wherein the reflector is constructed and arranged to direct a quantity of ultraviolet radiation from the ultraviolet radiation source that does not contact the doorknob back to the doorknob.
